(19) United States
(12) Patent Application Publication
(10) Pub. No.: US 2009/0264706 A1
(43) Pub. Date: Oct. 22, 2009

(54) Disposable interchangeable angle light guide system

(76) Inventor: John L. Bala, Pomfret Center, CT (US)

Correspondence Address: ALIX YALE & RISTAS LLP, 750 MAIN STREET, SUITE 1400, HARTFORD, CT, 06103, US

(21) Appl. No.: 12/386,143

(22) Filed: Apr. 14, 2009

Related U.S. Application Data
(60) Provisional application No. 61/124,406, filed on Apr. 16, 2008.

(51) Int. Cl. A61B 1/06

(52) U.S. Cl. 600/160

(57) ABSTRACT

An interchangeable light guide system employs a multiplicity of disposable light guides each configured to provide a pre-selected off-axis angle of observation. The light guides are slid over the shaft of an endoscope of the zero angle endoscope and optically coupled with the probe to provide the off-axis observation angle. The specific observation angles are defined by the angles of the refractive surfaces of a prism. The proximal end of each of the adapters includes a knob to facilitate rotation of the light guide about the optical axis of the endoscope probe to orient the off-axis observation angle at a selected angular direction about the probe axis. The light guides are low cost disposable devices.

### CROSS-REFERENCE TO RELATED APPLICATION

This application claims the priority of U.S. Provisional Patent Application No. 61/124,406 filed Apr. 16, 2008, the disclosure of which is incorporated in its entirety.

### BACKGROUND

This disclosure relates generally to endoscopes and more particularly to optics and other mechanisms provided to achieve off-axis angle illumination, imaging and sterility.

### SUMMARY

Briefly stated, an interchangeable angled light guide system for selectively converting a zero angle endoscope to an angled off-axis endoscope employs a multiplicity of angle light guides each having a pre-established angle. Each light guide comprises a sleeve slidably receivable over the illumination tube or shaft of the endoscope. The sleeve mounts a prism and an illumination light extender which encloses the distal end of the endoscope illumination tube and couples with the endoscope optics upon installation. The prism comprises a first surface disposed at an angle to the optical axis of the endoscope. A second surface of the prism is transverse to the optical axis. The sleeve is also freely rotatable about the optical axis to vary the observation direction of the selected observation angle of the endoscope.

In a preferred form, a light extender and prism is attached to an end of the tubular sleeve. A retainer composed of non-abrasive materials is received in the extender. The retainer receives and fixedly mounts the prism. The sets of light guides with different prism characteristic angles are provided. In one embodiment, the surfaces of the prism set are disposed at angles of 0, 15, 22.5, 30, and 45 degrees which are integrated with a light extender having the same angles. Other angles may also be employed. The light guide further preferably comprises a knob at the proximal end to rotate the prism about the longitudinal axis. The prism preferably has the form of a cylindrical wedge. The light guides are preferably disposable.

A method of changing the observation angle of a zero angle endoscope comprises selecting a light guide having a prism with a characteristic refractive angle for the desired observation angle and an integrated illuminating extender. The prism optically couples with the endoscope and the light extender. The light guide is slid over the shaft end of the endoscope. The light guide is also rotated to orient the angle of observation and illumination about the optical axis of the endoscope. Sets of multiple light guides may be provided to accommodate the differing shaft geometries of various endoscopes.

### BRIEF DESCRIPTION OF DRAWINGS

FIG. 1 is a schematic diagram of an interchangeable angle light guide system.

FIG. 2 is a longitudinal side view of a representative endoscope employing a representative interchangeable angle light guide for the system of FIG. 1.

FIG. 3 is a perspective view of an interchangeable light guide in a sterilized pre-usage packaged form;

FIG. 4 is a longitudinal side view, partly broken away, of a representative light guide.

FIG. 5A is a longitudinal side view, partly broken away and partly in section, of a representative light guide;

FIG. 5B is an enlarged sectional view of a left hand portion of the light guide of FIG. 5A;

FIG. 5C is an enlarged sectional view of a lower right hand portion of the light guide of FIG. 5A;

FIG. 5D is an enlarged end view looking from the right of the light guide of FIG. 5A;

FIG. 6A is an enlarged end view, partly in phantom, of a knob for the light guide of FIG. 5A;

FIG. 6B is an enlarged end perspective view of the knob of FIG. 6A taken from a generally opposite direction thereto;

FIG. 7A is a perspective view, partly in phantom, of a light guide extender employed in the light guide of FIG. 5A;

FIG. 7B is an enlarged side sectional view of the light guide extender of FIG. 7A;

FIG. 7C is an enlarged end view, partly in phantom, of the light guide extender of FIG. 7A.

FIG. 8A is a prism holder, partly in phantom, employed in the light guide of FIG. 5A;

FIG. 8B is a longitudinal side sectional view, partly in phantom, of the prism holder of FIG. 8A;

FIG. 8C is an end view, partly in phantom, of the prism holder of FIG. 8A.

FIG. 9A is an enlarged side view, partly in diagram form, of a prism employed with a light guide of FIG. 1;

FIG. 9B is an end view of the prism of FIG. 9A;

FIG. 10 is a side and end view composite, in diagram form, of a prism that may be employed with the angle light guide of FIG. 1;

FIG. 11A is a side view, partly in section, of a representative endoscope employing a second embodiment of an interchangeable angle light guide;

FIG. 11B is an enlarged fragmentary view of the guide and endoscope of FIG. 11A;

FIG. 12 is an optical diagram illustrating principles of the interchangeable angle light guide;

FIG. 13 is a side view, partly in section, of another embodiment of an interchangeable angle light guide for an endoscope;

FIG. 14 is a side elevational view of the endoscope and light guide of FIG. 13 illustrated in an operational configuration; and

FIG. 15 is an enlarged view, partly in section, partly in phantom and partly broken away, of an interchangeable angle light guide and a shaft end of an endoscope.

### DETAILED DESCRIPTION

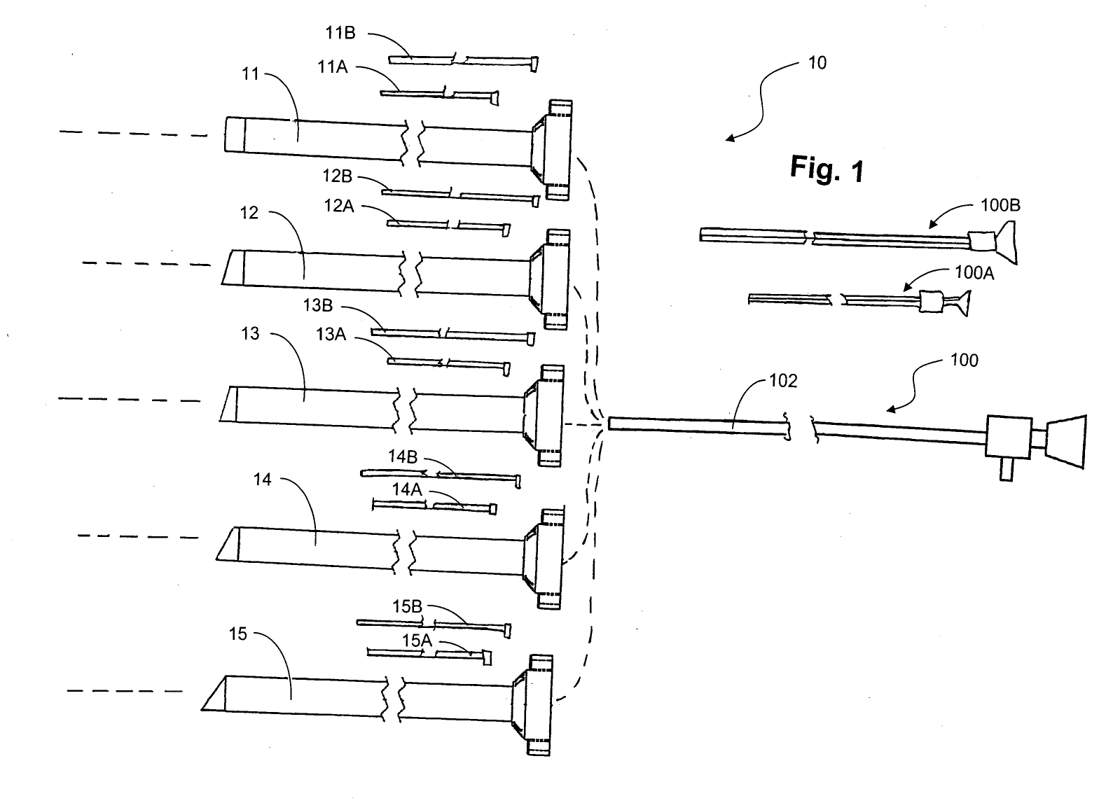

With reference to the drawings wherein like numerals represent like parts throughout the several Figures, an interchangeable light guide system is schematically illustrated in FIG. 1 and designated generally by the numeral 10. The light guide system functions to change the angle of illumination and observation at the probe or shaft end of a zero angle endoscope 100 in a highly efficient and low cost manner. The interchangeable angle light guide system employs several sets of multiple angled light guides. Each light guide of a set produces a unique pre-established angle. Each light guide is interchangeable to adapt a zero angle endoscope to operate at a selected off-axis observation angle as required for a given procedure. Each light guide is preferably disposable.

As illustrated in FIG. 1, five light guides designated generally by the numerals 11-15 are respectively configured to provide 0, 15, 22.5, 30, and 45 degree off-axis observation when installed over a zero angle endoscope shaft. It will be appreciated that neither the number of different angle light guides, nor the specific angles are limited to those described herein. In addition, it will be appreciated that the lengths of the light guides and their diameters are dimensioned for compatibility and use in conjunction with various specific pre-existing endoscopes. Light guides of a given angle for different endoscope dimensions and/or geometries are designed with letters corresponding to their compatible endoscope. For purposes of description a representative light guide is generally designated by the numeral 16.

In a preferred form, the light guides are manufactured from relatively low cost materials and are designed for a single procedure and then placed in waste disposal. Prior to usage the sterilized light guides 16 are placed in an individual package 18, such as shown in FIG. 3. The light guides are thus preferably disposable although in some embodiments they may be sterilized and used for multiple procedures. Once a specific off-axis angle of observation is desired, the light guide for the appropriate angle is removed from the package and slidably inserted over the probe 102 of the given endoscope. After usage, the light guide 16 may be removed from the probe end of the endoscope and placed in the package 18 or otherwise placed in condition for disposal. Given the disposability feature, the physician would typically have numerous multiple sets of light guides for different off-axis observation angles. In addition, the physician would have different multiple sets of light guides also for different endoscope shaft dimensions.

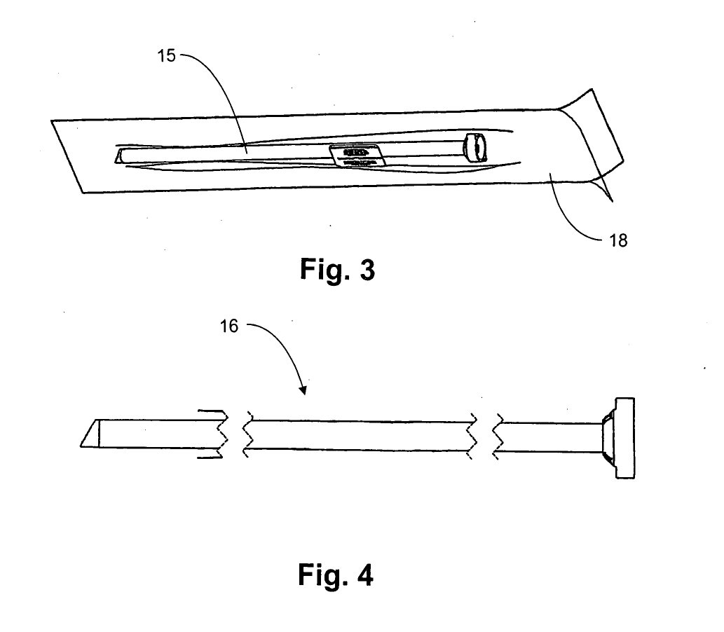

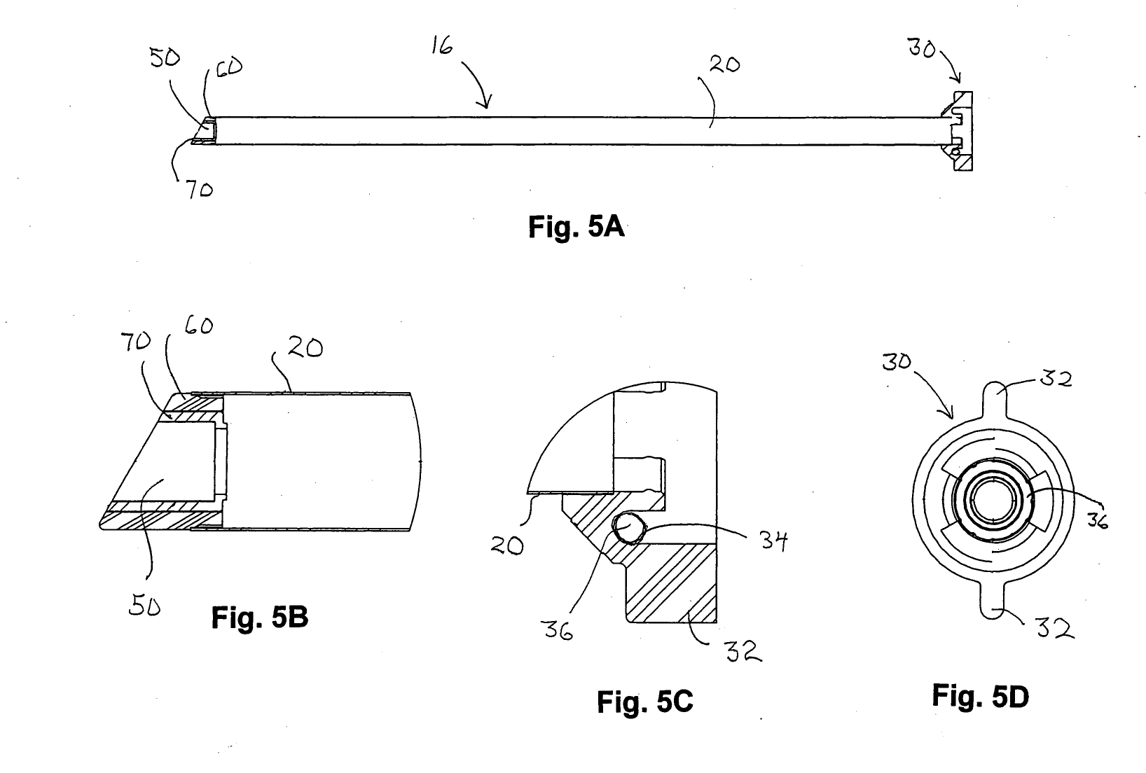

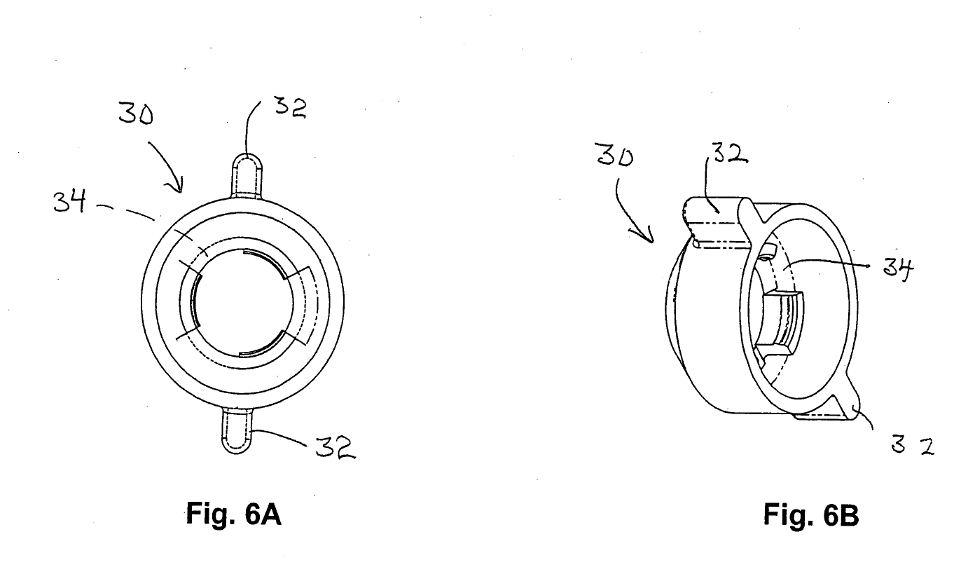

With reference to FIGS. 4 and 5A, each light guide 16 includes an elongated tube-like sleeve 20 made of polyetheretherketone (PEEK) material which has a length and diameter as may be required for the corresponding endoscope for which it is adapted. A flange-like knob 30 is mounted at the proximal end of the sleeve 20. The knob 30 integrally includes a pair of diametral projections 32 (FIGS. 5D, 6A and 6B) which facilitate manipulation of the light guide about the longitudinal axis (optical axis of the endoscope). The interior of the knob includes an annular groove 34 (FIGS. 5A, 5C and 6B) which receives a sealing ring 36. The knob is adapted to engage against the end of the endoscope and to seal with the illumination tube or the shaft. For example, for one embodiment of light guide 16 the length of the light guide 16 is 302.3 millimeters, the exterior diameter is 10.5 millimeters and the interior diameter is 4.0 millimeters. Naturally, other dimensional relationships may be provided as required.

The principal optical components for the light guide are a prism and light extender. The prism couples with the endoscope optics to produce the off-axis angle image and light extender for illumination that interfaces with the observation for the endoscope. The specific angle is established by the characteristic prism refractive angles and the illumination angles. For descriptive purposes, a representative prism, light guide extender and prism retainer are respectively designated by the numerals 50, 60, and 70.

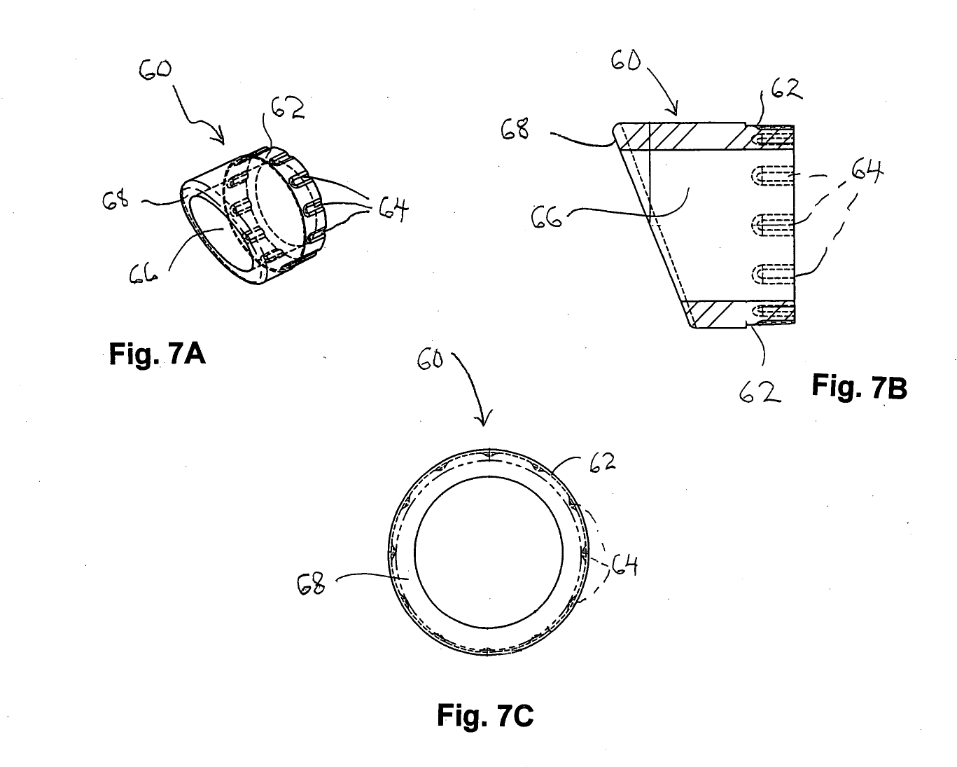

The distal end of the light guide includes the light guide extender 60 which receives the prism retainer 70 which, in turn, mounts the prism 50. Each of the foregoing components is dimensioned and configured for a given pre-established observation angle. With additional reference to FIGS. 7A-7C, the light guide extender 60 includes a slightly recessed annulus 62 which is traversed by a plurality of angularly spaced longitudinal depressions 64. The foregoing configuration facilitates receiving the end of the sleeve and securing same in a tight close fitting relationship. The extender 60 interiorly forms a cylindrical cavity 66 and frontally terminates in an obliquely oriented edge face 68 which is configured for the specific angle. The light guide extender 60 may be manufactured from Zeonex™ material or other light conducting plastics or glass.

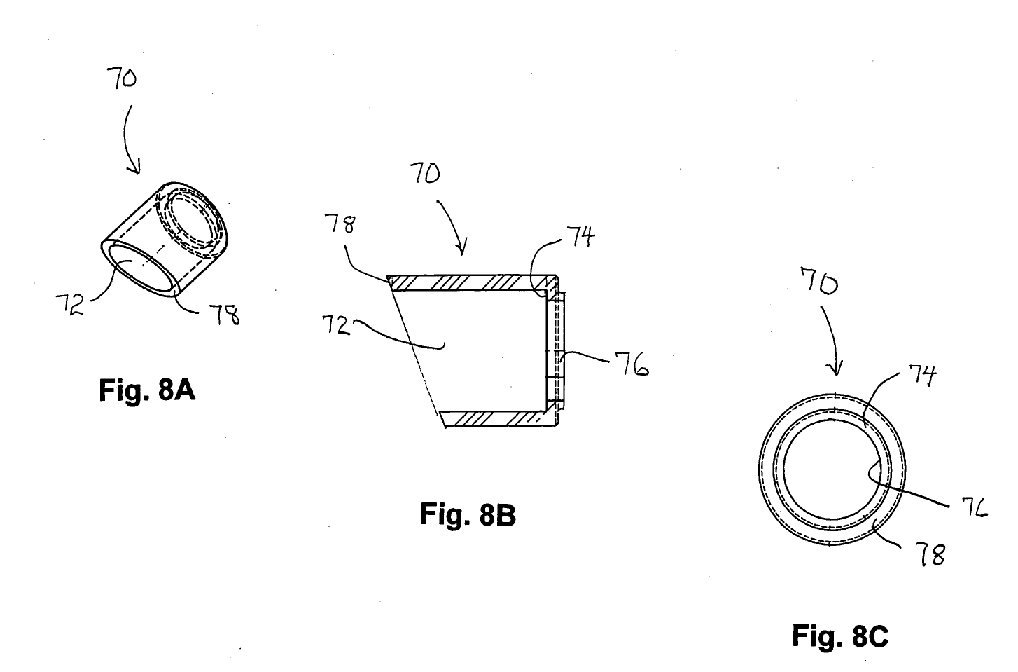

The prism holder of retainer 70 (FIGS. 8A-8C) is closely received in the cavity of the extender 60. The prism retainer 70 has a cylindrical cavity 72 with a proximal rim 74 and an enlarged axial aperture 76. The frontal edge face 78 is also disposed at the same angle as the extender edge face 68.

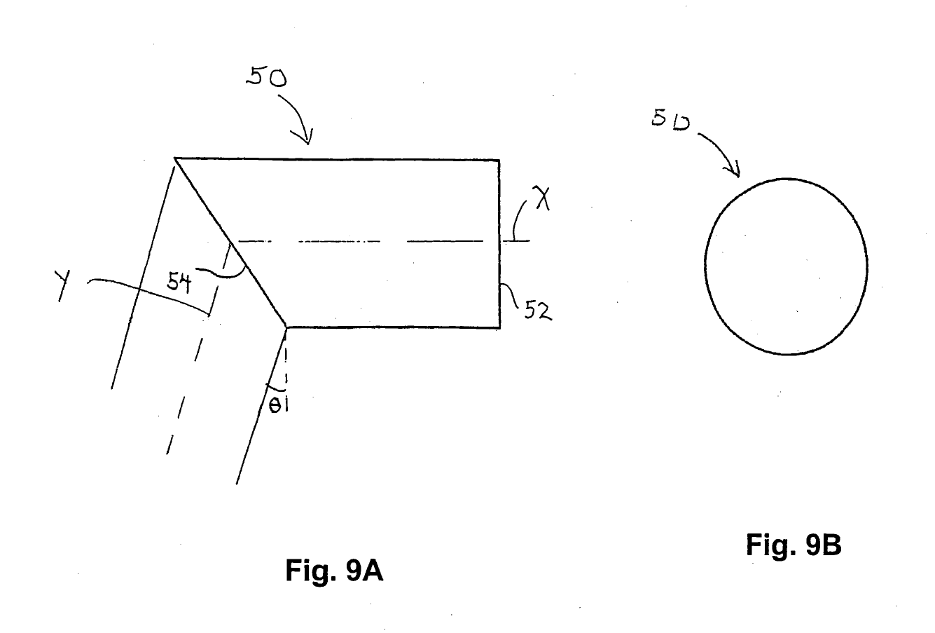

The prism retainer 70 closely receives and fixedly mounts the prism 50 (FIGS. 9A and 9B). The prism 50 is a cylindrical solid (cylindrical wedge) having a planar frontal face 54 obliquely oriented at an angle to the central longitudinal axis or optical axis X to define the angled off-axis Y for the light guide. The front end face 78 of the retainer is preferably co-planar to the frontal face 54 of the prism. The prism 50 is preferably dimensional to be fully received in the prism retainer 70. Preferably the retainer 70 is a black non-reflective material. Preferably the frontal prism surface 54 is planar. However, the surface 54 may also be convex or concave in some embodiments. Note the prism retainer can be eliminated using a prism in which the sides are blackened to serve the same purpose as the retainer with the use of an aperture.

The prism retainer 70 interlocks with the endoscope probe and, upon proper installation of the guide sleeve, is positioned relative to the optical channel and the objective lens of the endoscope to form a light trap thereby preventing stray illumination light from entering the optical channel which would reduce the resolution of the optic imaging system. The retainer may be secured in place by adhesive or other securement means. With reference to FIGS. 8A and 8C, the retainer 70 is preferably made of soft, plastic, non-abrasive material to prevent it from scratching the surface of the optical components and the objective lens and to improve the efficiency of the light trap.

In one embodiment illustrated in FIGS. 9A and 9B, the prism 50 is made of LAH58, LASFN9 or ZlaF68 high index glass material, has a diameter of 6.0 millimeters and a total axial length of 6.00 millimeters, although the diameter and length can be varied. As indicated, the prism may have an angle θ which is, for example, 0, 15, 22.5, 30 and 45 degrees. Other angles are possible. The prism essentially assumes the form of a cylindrical wedge. The characteristic angle for each light guide is defined by the refraction angle of the prismatic face 54. Other prism geometries are possible. The effective angle of the prism may also be adjusted by orienting the prism at an angle to the longitudinal axis of the light guide (and the associated endoscope shaft).

The light guide extender 60, prism holder 70 and prism 50 which are nested with each other impose a specific optical angle at the frontal face for each of the different desired off-axis observation angles. A zero angle configuration may also be provided. The zero angle light guide may function to maintain a sterile conduit for the endoscope.

In summary, each light guide functions to implement an off-axis angle of illumination and observation at the probe or illumination shaft end of a zero angle endoscope 20. The angle light guide system employs several sets of multiple angle light guides, each light guide producing a unique pre-established angle, and being interchangeable to provide a selected off-axis observation angle as required for a given procedure. Multiple light guide sets, for example, light guides 11A, 12A, 13A, 14A and 15A are provided for each given endoscope illumination shaft geometry, for example, for endoscope 100A.

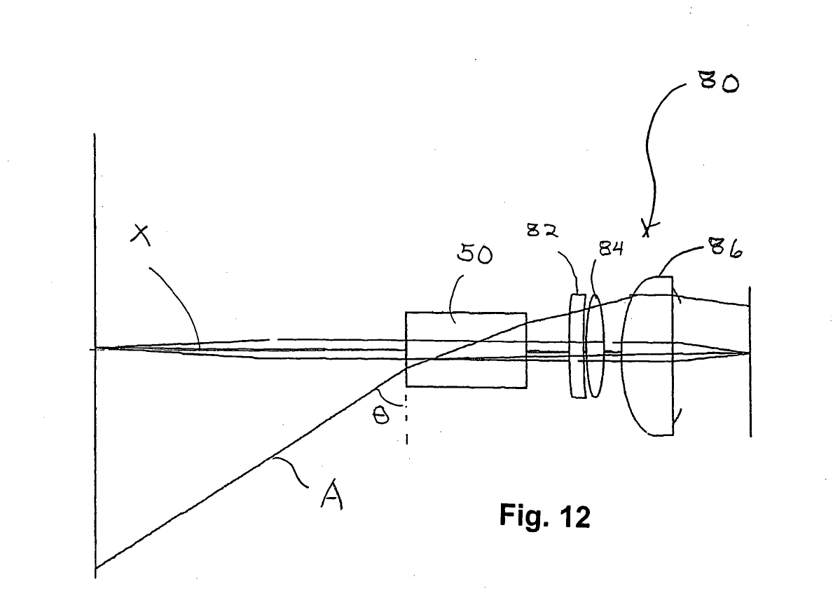

The optical characteristics of the light guides upon proper installation on a zero angle endoscope are illustrated with reference to FIG. 12. A current representative 6.25 millimeter objective designated generally by the numeral 80 may, for example, employ a convex/concave element 82, a biconvex element 84 and convexo/planar element 86 at the probe end of the endoscope (not illustrated). As illustrated, a prism 50 is positioned in front of the objective to provide an angular illumination/imaging angle θ for axis X in relation to the transverse plane to the longitudinal optical axis A of the endoscope probe. Prism 50 is schematically illustrated in FIG. 12 as a zero angle prism for purposes of illustration and further discussion.

In the present prismatic design, the change in the angle of light is accomplished by the refractive index variation and the angle θ characteristic of a prism face 54 interposed in the optical channel.

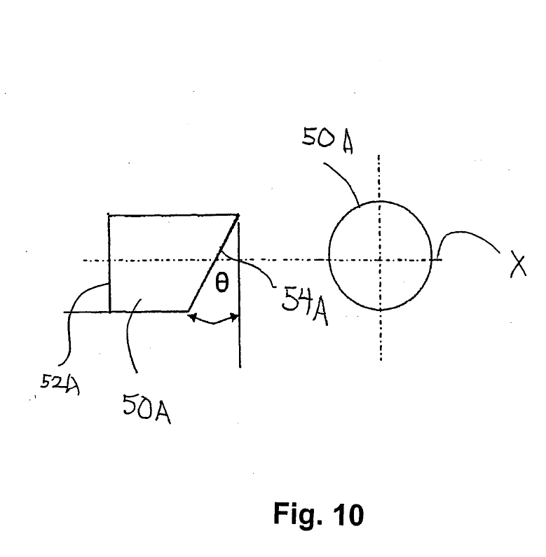

The prism 50A of FIG. 10 has a 6.25 mm diameter and a length of 9.00 mm. The prism is constructed of a very high index optical glass such as H-Z1aF68 material. The prism 50A is ground and polished to a half fringe irregularity and has a surface marked 40-20-S-D with an 80% clear aperture. Both surface 52A and surfaces 54A are coated with an anti-reflection coating.

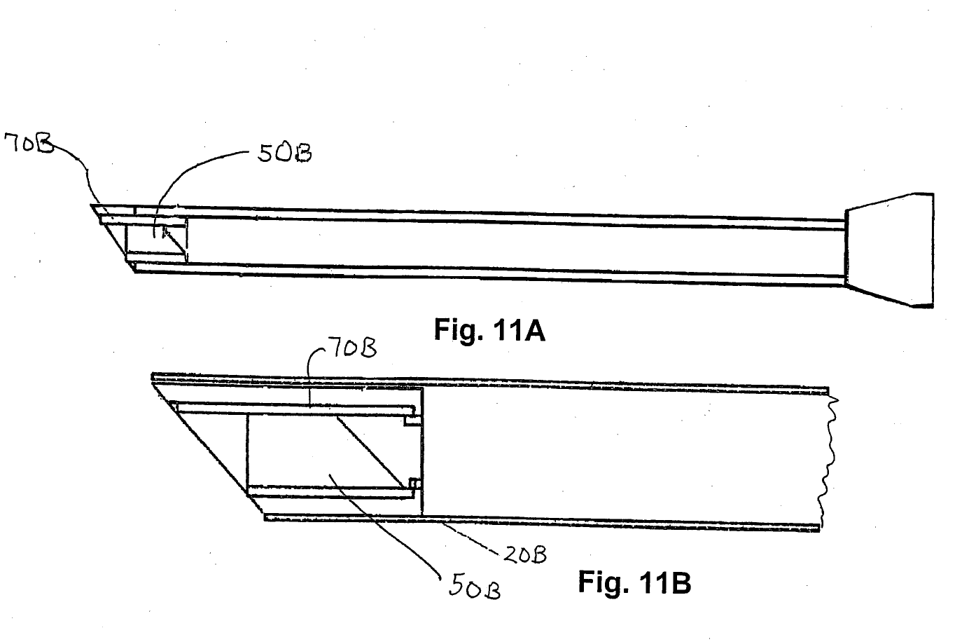

With reference to FIGS. 11A and 11B, instead of the prism 50B presenting a face at the selected angle at the front surface 52B, the prism front surface 52B is essentially symmetric about optical axis X (planar, or convex or concave) and the path of the light is refracted at the various angles of the rear surface 54B of the prism.

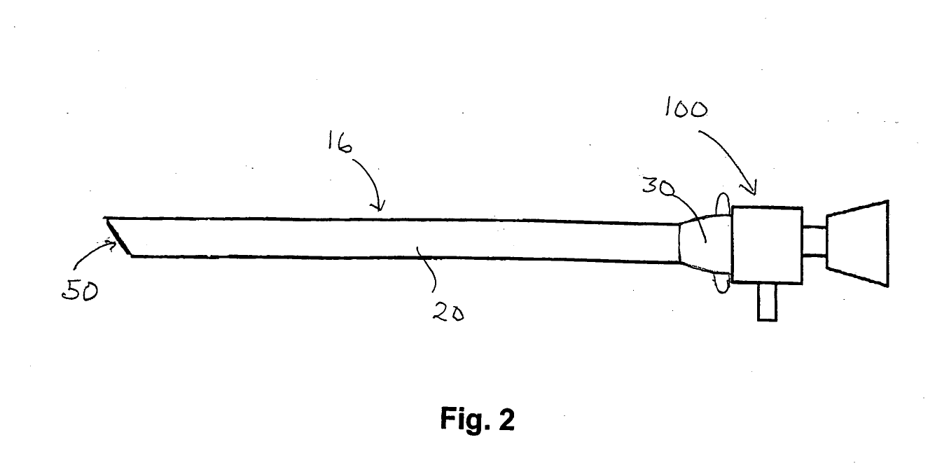

As illustrated in FIG. 2, the rotating knob 30 is disposed at the proximal end of the light shaft in fixed rotational relationship with the sleeve 20 and the prism 50. The knob 30 is employed to rotate the prism 50 to thereby rotate the image about the principal optical axis X of the distal end of the endoscope. By contrast, with current dedicated angular viewing endoscopes to which the disclosure relates, any rotation also rotates the endoscope itself including the fiber optic light cable and the electronic camera cables.

It should also be appreciated that a condom (not illustrated) maybe used similar to those for the zero degree light guide to maintain the scope, camera and endo-coupler in a sterile environment.

With reference to FIG. 1 the interchangeable light guide system 10 employs multiple sets of light guides 11-15 which are adapted to mount and optically couple with a conventional zero angle endoscope 100. Each light guide 11,11A 11B is also appropriately dimensioned and configured for installation with the respective conventional endoscope 100, 100A, 100B to convert from a zero degree viewing scope to a selected angle as desired by the physician. The series of adaptive light guides 11-15, 11A-15A, 11B-15B, which differ only in the prismatic angle and length for each configuration are interchangeable in real time. In place of the dedicated scopes with complete optical systems designed for a given angle, each light guide converts a zero degree observation to a versatile angled scope with the implementation of the specifically configured and oriented prism and light extender as previously described. The set of light guides thus converts a zero degree viewing scope to a multiplicity of angled scopes in a highly efficient and low cost manner. Each endoscope illumination shaft configuration may require its own set of light guides.

In one embodiment an interchangeable angle light guide includes plastic sleeve-like tube which extends the length of the shaft functions to contain a plastic light extender as a conduit which transmits the light from the distal end of an endoscope to illuminate the field of view related to the image angle.

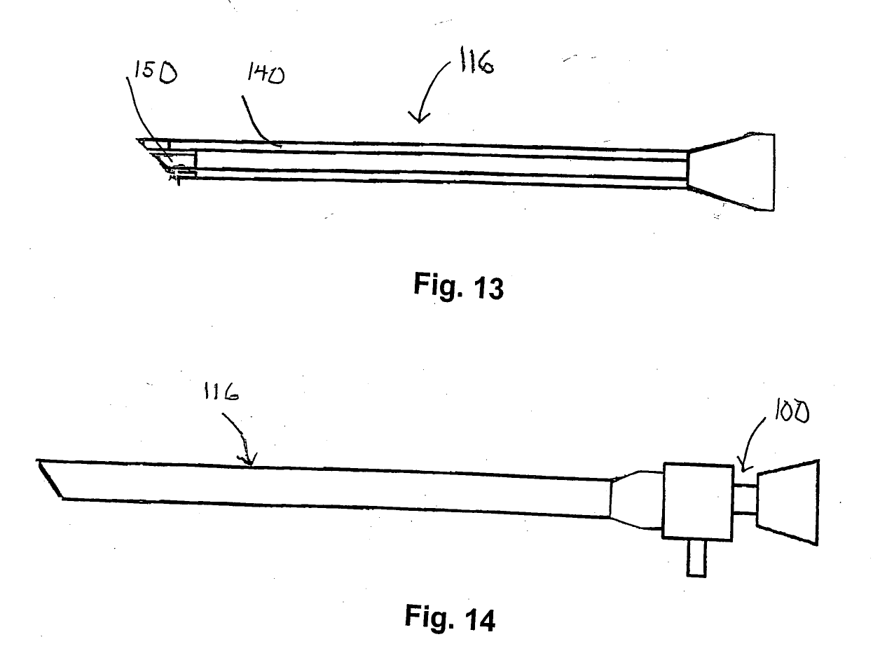

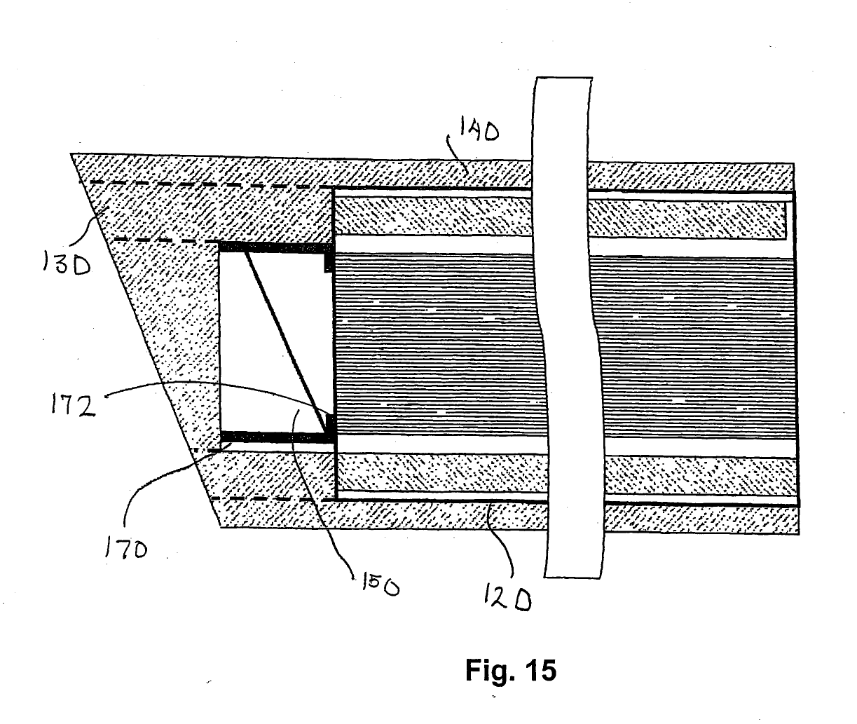

With reference to FIGS. 13-15, the light guide 116 primarily differs from the light guide 16 in the sleeve 140 which mounts the prism package and illumination extender and slides over the tubular shaft 120. The fiber optic extender 130 at the distal end of the endoscope serves to couple light from the distal end of an endoscope which serves as the annulus which becomes a shortened light guide. The prism 150 and optics at the distal end of the device are essentially the same as previously described. The plastic sleeve may be replaced by individual fibers. However, in the preferred embodiment, a plastic light guide would be recommended to eliminate the potential heat build up in the distal end of the device caused by misalignment of the fibers. This also tends to make the device lower in cost and therefore more disposable. The sleeve 140 may be stainless steel or plastic. The retainer 170 forms an aperture 172 at the frontal face of the optical channel.

1. An endoscope adaptor comprising:
an elongated zero angle optical probe shaft having a distal end and a proximal end and defining a longitudinal optical axis; and
a light guide comprising a sleeve slidably receivable over said shaft and mounting a prism assembly which encloses the distal end of said shaft and optically couples with said shaft, said prism assembly comprising a prism having a first surface disposed at a first angle to said optical axis and having a second surface transverse to the optical axis to produce an observation axis at an observation angle to said shaft optical axis and a light extender having the same first angle to extend an illumination path, and said sleeve being rotatable about said optical axis to vary the angular direction of the observation angle.

2. The endoscope adaptor of claim 1 wherein said light extender is fixedly attached to an end of said sleeve and a retainer is received in said extender and fixedly mounts said prism.

3. The endoscope adaptor of claim 1 further comprising a knob at the proximal end of said sleeve to facilitate rotation the prism about the longitudinal optical axis.

4. The endoscope adaptor of claim 3 wherein said knob comprises a diametral pair of tabs.

5. The endoscope adaptor of claim 1 wherein said first surface of said prism is disposed at an angle selected from the group consisting of 0, 15, 22.5, 30 and 45 degrees.

6. A light guide assembly for a zero angle endoscope comprising:
a sleeve having a distal end and a proximal end and forming a light channel defining a longitudinal axis;
a knob disposed at said proximal end; and
a prism assembly interposed along said longitudinal axis adjacent said distal end and comprising a prism having a first planar surface which forms an angle with an orthogonal plane to said longitudinal axis and a longitudinally spaced second surface oriented substantially orthogonally to said longitudinal axis.

7. The light guide assembly of claim 6 wherein said first planar surface of said prism has an angle selected from the group consisting of 0, 15, 22.5, 30 and 45 degrees.

8. The light guide assembly of claim 6 wherein said second surface has a shape selected from the group consisting of planar, concave and convex.

9. The light guide assembly of claim 6 wherein said prism has the form of a cylindrical wedge.

10. The light guide assembly of claim 6 wherein said prism is constructed from material selected from the group consisting of LAH58 glass, LASFN9 glass, ZlaF68 glass and high index glass.

11. The light guide assembly of claim 6 wherein said sleeve is constructed from material selected from the group consisting of polyetheretherketone (PEEK) material, plastic material, metal and glass.

12. A method of changing the observation angle of a zero angle endoscope having a shaft defining an optical axis comprising:
selecting a light guide having a prism corresponding to the desired observation angle;
sliding the light guide over the shaft of the endoscope;
optically coupling the light guide prism with the shaft; and
optically coupling light from the endoscope with the light guide for illumination.

13. The method of claim 12 further comprising rotating the light guide to orient the angle of observation about the optical axis of the probe.

14. The method of claim 12 further comprising axially removing the light guide from the probe and installing a second light guide on the probe.

15. The method of claim 14 further comprising disposing of the removed light guide.

16. The method of claim 10 further comprising combining illumination light from the endoscope while simultaneously receiving light into the endoscope.

17. The light guide of claim 6 wherein the prism has a longitudinal axis and the prism is positioned so that its longitudinal axis is at an angle to the longitudinal axis of said light guide.

18. The endoscope adaptor of claim 1 wherein the prism has a longitudinal axis and the prism is positioned so that its longitudinal axis is at an angle to the longitudinal axis of said light guide.

19. The endoscope adaptor of claim 1 wherein the prism is constructed of a glass having a high index of refraction.

20. The endoscope adaptor of claim 1 wherein the sleeve has a thin wall and is constructed from polyetheretherketone (PEEK) material.
